(19) United States
(12) Patent Application Publication
(10) Pub. No.: US 2018/0271387 A1
(43) Pub. Date: Sep. 27, 2018

(54) SYSTEM AND METHOD FOR HEART RATE MEASUREMENT

(71) Applicant: Renesas Electronics Corporation, Tokyo (JP)

(72) Inventor: Koji NISHIGAKI, Tokyo (JP)

(73) Assignee: Renesas Electronics Corporation, Tokyo (JP)

(21) Appl. No.: 15/857,759

(22) Filed: Dec. 29, 2017

(30) Foreign Application Priority Data
Mar. 22, 2017 (JP) 2017-055470

(51) Int. Cl. A61B 5/024; A61B 5/22; A61B 5/0245

(52) U.S. Cl. CPC A61B 5/02405; A61B 5/222; A61B 5/7275; A61B 5/02438; A61B 5/02427; A61B 5/0245

(57) ABSTRACT

The heart rate of a subject can be measured on a regular basis during the subject's daily performance without causing the subject to take trouble to wear a special device or to feel bothersome to keep wearing a special device. A heart rate measurement system includes an intraoral electrode, a hand electrode, and a detection unit. The intraoral electrode is installed in a head portion with implanted brush hair of a toothbrush and comes into contact with an intraoral region of the subject. The hand electrode is installed in a grip of the toothbrush and comes into contact with the subject's hand. The detection unit detects the subject's heart rate. Based on signals from the intraoral electrode and hand electrode, the detection unit derives a potential difference between the intraoral region and the hand and, based on the potential difference, collects heart rate data on the subject.

### CROSS-REFERENCE TO RELATED APPLICATIONS

The disclosure of Japanese Patent Application No. 2017-055470 filed on Mar. 22, 2017 including the specification, drawings and abstract is incorporated herein by reference in its entirety.

### BACKGROUND

The present invention relates to a heart rate measurement system, for example, a heart rate measurement system in which a potential difference between two parts of the body of a subject is measured and the heart rate of the subject is determined based on the potential difference.

An electrocardiograph has been known which is used to measure an electrocardiographic waveform of a subject by putting a positive electrode and a negative electrode in contact with predetermined body parts of the subject. In Japanese Unexamined Patent Application Publication No. 2009-131461, an electrocardiograph is disclosed which includes a main unit provided with an electrode to be put in contact with a hand of a subject and an external electrode unit provided with an electrode coupled with the main unit via a cable and to be put in contact with the chest of the subject.

### SUMMARY

In recent years, attempts of disease predictive diagnosis are being made in which live-body cardiovascular data, for example, an electrocardiogram of a subject is generated on a regular basis during daily performance of the subject and the data generated is used for disease predictive diagnosis on the subject, for example, to determine the likelihood of developing a lifestyle-related disease. The electrocardiograph disclosed in Japanese Unexamined Patent Application Publication No. 2009-131461, however, requires the subject to take trouble to wear the device and causes the subject to feel bothersome to keep wearing the device. Besides, since the measurement performed using the electrocardiograph is not a part of the subject's daily performance, the subject tends to forget to perform measurement using the electrocardiograph, so that collecting live-body data on the subject on a regular basis is difficult.

Other objects and novel features of the present invention will become clear from the following description and the attached drawings.

According to an embodiment of the present invention, a heart rate measurement system includes an intraoral electrode, a hand electrode, and a detection unit. The intraoral electrode is installed in a head portion where brush hair is implanted of a toothbrush and comes into contact with an intraoral region of a subject. The hand electrode is installed in a grip of the toothbrush and comes into contact with a hand of the subject. The detection unit detects a heart rate of the subject. In the heart rate measurement system, the detection unit derives, based on signals from the intraoral electrode and the hand electrode, a potential difference between the intraoral region and the hand of the subject and, based on the potential difference derived, collects heart rate data on the subject.

According to the above embodiment, the subject can measure the heart rate on a regular basis during the daily performance without taking trouble to wear a special device and without feeling bothersome to keep wearing such a special device.

### BRIEF DESCRIPTION OF THE DRAWINGS

FIG. 1 is a schematic diagram showing an example configuration of a heart rate measurement system according to an embodiment outline.

FIG. 2 is a schematic diagram showing an example configuration of a heart rate measurement system according to a first embodiment.

FIG. 3 is a view on arrow A in FIG. 2.

FIG. 4 is a view on arrow B in FIG. 2.

FIG. 5 shows an example circuit which is included in a detection unit of the heart rate measurement system according to the first embodiment and which is used to detect the potential difference between an intraoral electrode and a hand electrode.

FIG. 6 is a flowchart of processing performed in the heart rate measurement system according to the first embodiment.

FIG. 7 is a schematic diagram showing an outline configuration of a hand electrode according to a modification example 1.

FIG. 8 is a schematic diagram showing the configuration of a heart rate measurement system according to a second embodiment.

FIG. 9 is a diagram for describing how the condition of subject's blood vessels is determined based on heart rate data and pulse data.

FIG. 10 is a flowchart of processing performed in a heart rate measurement system according to a second embodiment.

FIG. 11 is a schematic diagram showing the configuration of a heart rate measurement system according to a third embodiment.

FIG. 12 is a view on arrow C in FIG. 11.

FIG. 13 is a view on arrow D in FIG. 11.

FIG. 14 is a schematic diagram showing a water path included in the heart rate measurement system according to the third embodiment.

FIG. 15 is a view on arrow E in FIG. 14.

### DETAILED DESCRIPTION

In the following, embodiments of the present invention with means for solving the above problem applied to will be described in detail with reference to drawings. To make description clear, the following description and drawings will include omission and simplification as considered appropriate. Also, elements shown in the following drawings as functional blocks to perform various processing can be configured hardware-wise, for example, by a CPU (Central Processing Unit), memory or other circuits and software-wise, for example, by programs loaded in a memory. Therefore, it is readily understandable by those skilled in the art that such functional blocks can be configured in various forms, for example, by hardware only, by software only or by a combination of hardware and software without being limited to any one form. Also, in the following drawings, identical elements are denoted by identical symbols and duplicate description is omitted as considered appropriate.

For loading into a computer, the programs mentioned above can be stored in various types of non-transitory computer-readable media. The non-transitory computer-readable media include various types of tangible storage media such as magnetic recording media (e.g., flexible disks, magnetic tapes and hard disks), magneto-optical media (e.g., magneto-optical disks), CD-ROMs (Read Only Memories), CD-Rs, CD-R/Ws, and semiconductor memories (e.g., mask ROMs, PROMs (Programmable ROMs), EPROMs (Erasable PROMs), flash ROMs, and RAMs (Random Access Memories)). The programs may also be loaded into a computer using transitory computer-readable media, for example, electrical signals, optical signals and electromagnetic waves. The transitory computer-readable media can be used to load programs into a computer via wired communication paths such as electrical wires and optical fibers or wireless communication paths.

In the following, the description of the invention will be divided into two or more sections or will range over two or more embodiments as required for the sake of convenience. Unless otherwise expressed, such sections and embodiments are not mutually irrelevant. For example, among such sections and embodiments, one is a partial or total modification of another, one represents an application of another, or one elaborates or supplements another. Also, numbers referred to in the following description of embodiments (for example, numbers representing counts, numerical values, volumes, or ranges) do not represent defined values, that is, they may be smaller or larger unless otherwise expressed or except when they are apparently defined in principle.

Furthermore, the constituent elements (including operation steps) of the following embodiments are not necessarily indispensable unless otherwise expressed or except when they are considered apparently indispensable in principle. Similarly, the shapes of and positional relationships between constituent elements referred to in describing the following embodiments are inclusive of those substantially close to or similar to them unless otherwise expressed or except when such shapes and positional relationships are apparently considered strictly defined in principle. This also applies to the numbers (for example, numbers representing counts, numerical values, volumes, or ranges).

Outline of Embodiments

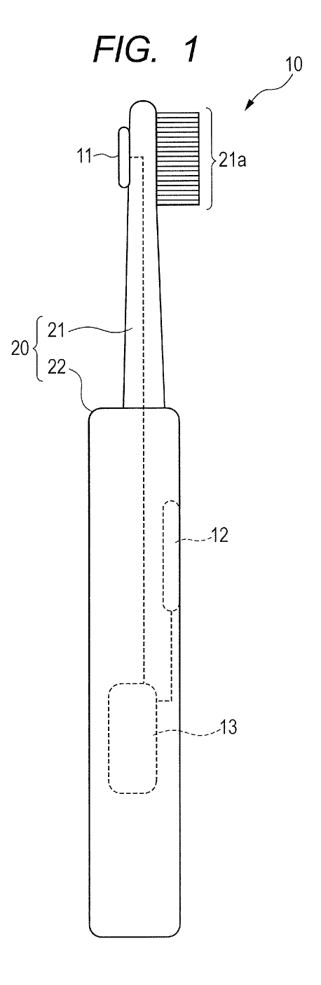

Before describing embodiments of the present invention in detail, an embodiment outline will be described below. FIG. 1 is a schematic diagram showing an example configuration of a heart rate measurement system 10 according to the embodiment outline. As shown in FIG. 1, the heart rate measurement system 10 is installed in a tooth brush 20 which includes a head portion 21 and a grip 22 with the head portion 21 including implanted brush hair 21a. The heart rate measurement system 10 includes an intraoral electrode 11, a hand electrode 12 and a detection unit 13.

The intraoral electrode 11 is attached to the head portion 21 of the toothbrush 20 and is to come into contact with an intraoral region of a subject. The hand electrode 12 is installed in the grip 22 and is to come into contact with a hand of the subject. The detection unit 13 is for detecting the heart rate of the subject. To be specific, the detection unit 13 derives, based on signals from the intraoral electrode 11 and the hand electrode 12, a potential difference between the intraoral region and the hand of the subject and, based on the potential difference derived, collects heart rate data on the subject.

As described above, the heart rate measurement system 10 is installed in the toothbrush 20 including the head portion 21 and the grip 22. While brushing the teeth in daily performance, the subject can collect the heart rate data using the intraoral electrode 11 and the hand electrode 12 both included in the heart rate measurement system 10 with the intraoral electrode 11 attached to the head portion 21 and the hand electrode 12 installed in the grip 22. This makes it possible for the subject to measure the heart rate on a regular basis through the daily performance without taking trouble to wear a special device and without feeling bothersome to keep wearing such a special device.

### First Embodiment

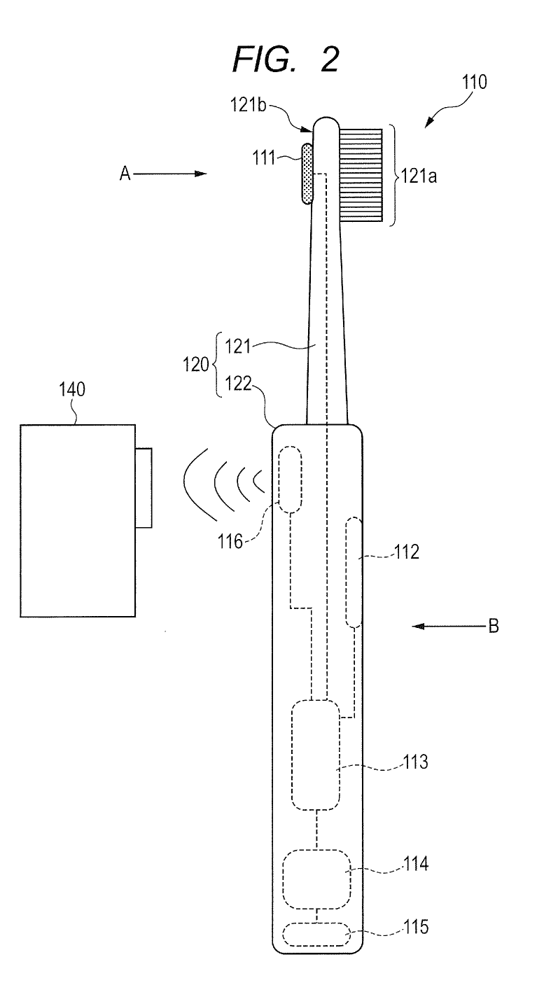

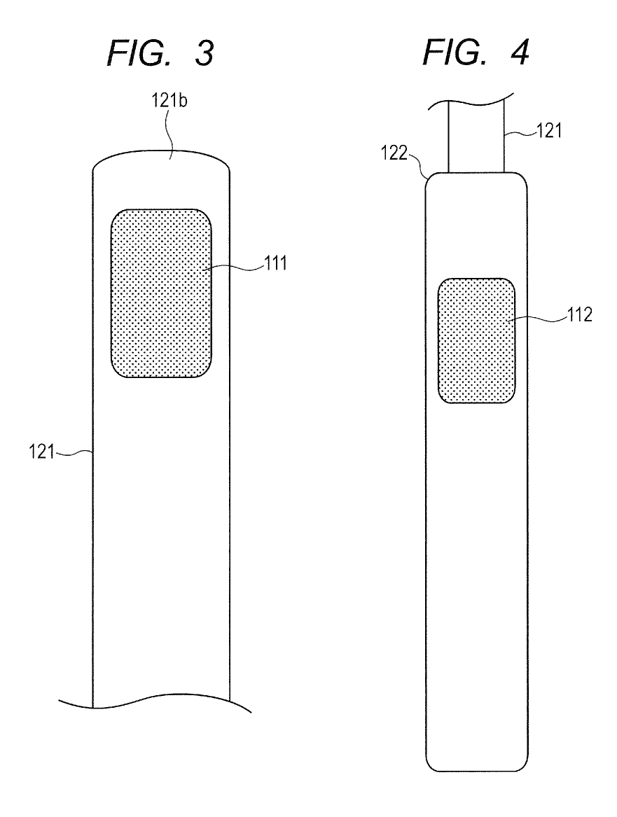

Next, a first embodiment of the present invention will be described in detail. FIG. 2 is a schematic diagram showing an example configuration of a heart rate measurement system 110 according to a first embodiment. FIG. 3 is a view on arrow A in FIG. 2. FIG. 4 is a view on arrow B in FIG. 2.

As shown in FIG. 2, the heart rate measurement system 110 is installed in a toothbrush 120 which includes a head portion 121 and a grip 122 with the head portion 121 including implanted brush hair 121a. The toothbrush 120 may be an electric toothbrush or a common non-electric toothbrush. The heart rate measurement system 110 includes an intraoral electrode 111, a hand electrode 112, a detection unit 113, a battery 114, a power receiving coil 115, a transmission unit 116, and an external system 140.

As shown in FIGS. 2 and 3, the intraoral electrode 111 is a plate-like electrode to come into contact with an intraoral region of a subject and is attached to a side 121b opposite to the other side where brush hair 121a is implanted of the head portion 121. With the intraoral electrode 111 configured as described above, when the head portion 121 is inserted in the oral cavity of the subject, the intraoral electrode 111 can be brought into satisfactory contact with the skin in the oral cavity of the subject. Also, as shown in FIGS. 2 and 4, the hand electrode 112 is installed in the grip 122 so as to come in contact with the hand of the subject. The hand electrode 112 is, therefore, to be installed in a portion of the grip 122 to come into contact with the hand of the subject when the subject brushes the teeth.

Referring to FIG. 2, the detection unit 113 derives a potential difference between an intraoral region and the hand of the subject based on signals from the intraoral electrode 111 and the hand electrode 112 and collects heart rate data on the subject based on the potential difference derived. The power receiving coil 115 is for contactlessly supplying power to the battery 114. The transmission unit 116 is for transmitting data (heart rate data in the present case) collected by the detection unit 113 to the external system 140, for example, by radio communication. The external system 140 is for analyzing the heart rate data received from the transmission unit 116. The head portion 121 and the grip 122 of the toothbrush 120 may be coated with a water-repellent material, for example, fluorine resin. Such coating will prevent the intraoral electrode 311 and the hand electrode 112 from conducting to each other via water. Also, using a primary cell, for example, a dry cell as the battery 114 makes it possible to configure the heart rate measurement system 110 without the power receiving coil 115.

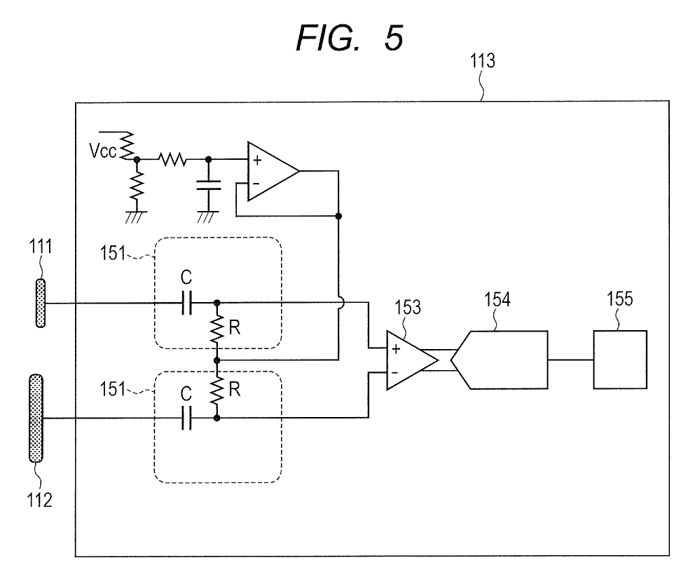

FIG. 5 shows an example circuit included in the detection unit 113 for detecting the potential difference between the intraoral electrode 111 and the hand electrode 112. As shown in FIG. 5, signals from the intraoral electrode 111 and the hand electrode 112 respectively pass through high-pass filters 151 each including a resistor R and a capacitor C and thereby have their noise contents suppressed. The signals are then led to a differential amplifier 153 where the potential difference between the intraoral electrode 111 and the hand electrode 112 is detected. An analog signal representing the potential difference detected at the differential amplifier 153 is sent to an AD converter 154 where the analog signal is converted into a digital signal. At a signal processing unit 155, the digital signal undergoes frequency analysis and heart rate data is calculated. The signal processing unit 155 includes an MCU (microcontroller unit) and a memory.

Next, the flow of processing in the heart rate measurement system 110 will be described below.

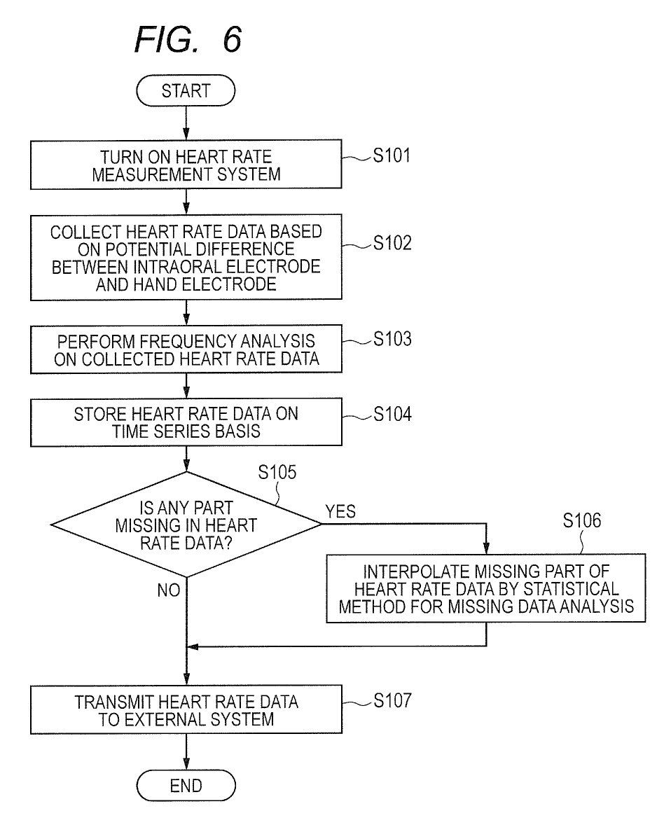

FIG. 6 is a flowchart of processing performed in the heart rate measurement system 110. As shown in FIG. 6, first, the heart rate measurement system 110 is turned on (S101). Next, in the detection unit 113, heart rate data is collected based on the potential difference between the intraoral electrode 111 and the hand electrode 112 (S102). To collect the heart rate data, the subject stays still for a predetermined amount of time (e.g., 5 to 10 seconds) with the intraoral electrode 111 kept in contact with an intraoral region of the subject. Then, in the detection unit 113, the heart rate data collected (S103) undergoes frequency analysis and is then stored on a time-series basis in a storage medium, for example, a memory included in the signal processing unit 155 (S104).

Subsequent to S104, whether any part is missing in the heart rate data stored is determined in the detection unit 113 (S105). When it is determined in S105 that there is a missing part in the heart rate data (YES), the missing part of the heart rate data is interpolated by a statistical method for missing-data analysis (S106). The statistical method for missing-data analysis used to analyze the missing part of the heart rate data may be a known one. For example, the value of a missing data part may be estimated based on the periodicity of the heart rate data. The transmission unit 116 transmits the heart rate data to the external system 140 (S107). When, in S105, it is determined that no part is missing in the heart rate data (NO), processing advances to S107.

As described above, the heart rate measurement system 110 is installed in the toothbrush 120 that includes the head portion 121 and the grip 122 and, in the system, heart rate data on a subject is collected using the intraoral electrode 111 attached to the head portion 121 and the hand electrode 112 installed in the grip 122 while the subject is brushing the teeth in daily performance. Namely, according to the heart rate measurement system 110, heart rate data on a subject is collected every time the subject brushes the teeth. Thus, the heart rate of the subject can be measured on a regular basis through the daily performance without making them take trouble to wear a special device and without making them feel bothersome to keep wearing such a special device.

In the heart rate measurement system 110, when any part is missing in the heart rate data collected, the missing part of the heart rate data is interpolated by a statistical method for missing-data analysis. In this way, even if, during heart rate measurement, data collection fails for a certain period, for example, due to poor contact between the intraoral electrode and an intraoral region of the subject or between the hand electrode and the subject's hand, the heart rate measurement need not be performed all over again. Therefore, the subject can measure the heart rate in a simpler and easier manner.

### MODIFICATION EXAMPLE 1

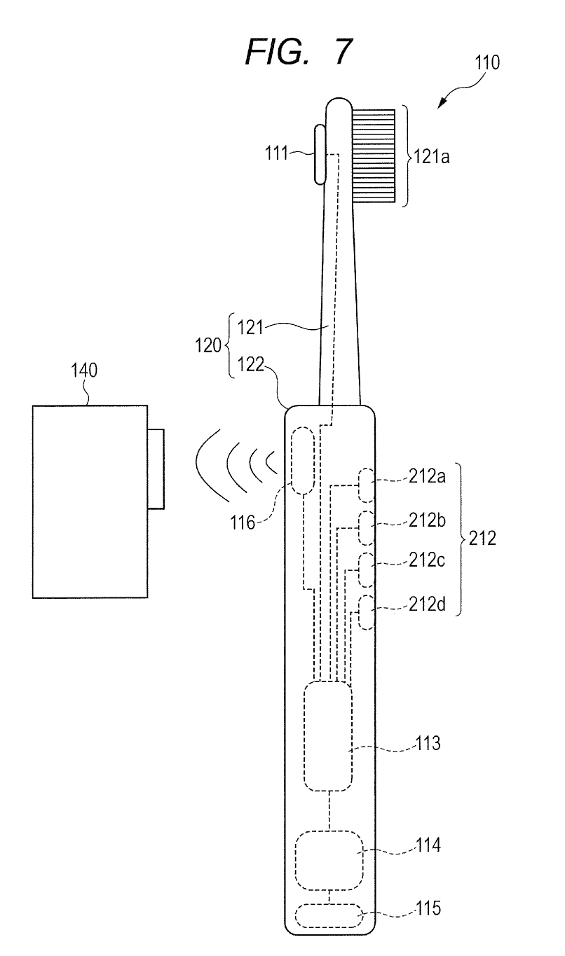

FIG. 7 is a schematic diagram showing an outline configuration of a hand electrode 212 different from the hand electrode 112 shown in FIGS. 2 and 3. As shown in FIG. 7, the hand electrode 212 includes mutually insulated split electrodes 212a, 212b, 212c, and 212d. The split electrodes 212a, 212b, 212c, and 212d are each independently coupled to the detection unit 113. The detection unit 113 collects heart rate data on a subject based on, out of the signals received from the split electrodes 212a, 212b, 212c, and 212d, the signal from which the largest potential difference between an intraoral region of the subject and the subject's hand has been derived.

In this way, heart rate data with no missing part on the subject can be collected when at least one of the split electrodes 212a, 212b, 212c, and 212d is in satisfactory contact with the subject's hand. For example, even in cases where some of the split electrodes 212a, 212b, 212c, and 212d are short-circuited with the intraoral electrode 111 via water, heart rate data with no missing part on the subject can be collected only if at least one of the split electrodes is in satisfactory contact with the subject's hand. This prevents occurrence of a case in which heart rate measurement on the subject has to be performed all over again and allows the subject to measure the heart rate in a simpler and easier manner.

### Second Embodiment

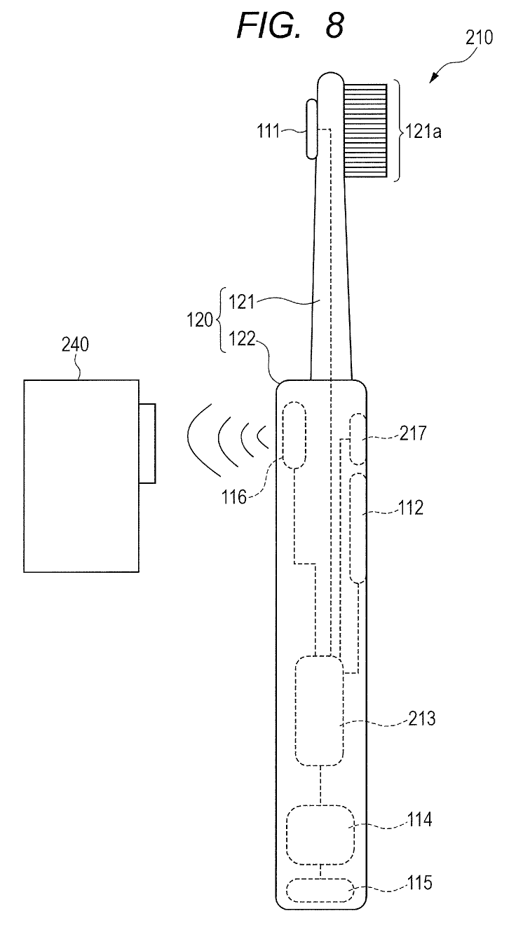

The configuration of a heart rate measurement system 210 according to a second embodiment will be described with reference to FIG. 8. In the following, description will be focused on aspects of the second embodiment differing from the first embodiment and the description of configuration aspects similar to those of the first embodiment will be omitted. FIG. 8 is a schematic diagram showing the configuration of the heart rate measurement system 210 according to the second embodiment. As shown in FIG. 8, like the heart rate measurement system 110 (see FIG. 2) according to the first embodiment, the heart rate measurement system 210 is installed in a tooth brush 120 which includes a head portion 121 and a grip 122. The heart rate measurement system 210 includes an intraoral electrode 111, a hand electrode 112, a detection unit 213, a battery 114, a power receiving coil 115, a transmission unit 116, an external system 240, and an optical sensor 217. Namely, relative to the heart rate measurement system 110 according to the first embodiment, the heart rate measurement system 210 according to the second embodiment additionally includes the optical sensor 217.

The optical sensor is for collecting heart rate data on a subject making use of the light-absorbing behavior of blood hemoglobin. The optical sensor 217 is installed in a portion of the grip 122 to come into contact with a finger of the subject when the subject brushes the teeth and includes a light source, for example, an LED (Light Emitting Diode) and a light receiving element (photodiode). The optical sensor 217 emits light from the light source to blood vessels inside the finger skin of the subject and detects, at the light receiving element, the amount of light (amount of reflected light) reflected from the blood vessels without being absorbed by blood hemoglobin.

The detection unit 213, like the detection unit 110 according to the first embodiment, derives a potential difference between an intraoral region of the subject and the subject's hand based on signals from the intraoral electrode 111 and the hand electrode 112 and collects heart rate data on the subject based on the potential difference derived. Furthermore, the detection unit 213 collects pulse data on the subject based on the amount of the reflected light detected by the optical sensor 217. The amount of the reflected light detected by the optical sensor 217 varies with the variation of blood amount in blood vessels (i.e. the amount of hemoglobin). Therefore, the detection unit 213 collects pulse data on the subject by monitoring fine variation in the amount of reflected light detected by the optical sensor 217.

The external system 240 is for analyzing the heart rate data and pulse data received from the transmission unit 116 and determining the condition of the subject's blood vessels. In the external system 240, variation of the condition of subject's blood vessels is detected based on the time difference between a peak of the heart rate data received from the transmission unit 116 and the corresponding peak of the pulse data also received from the transmission unit 116.

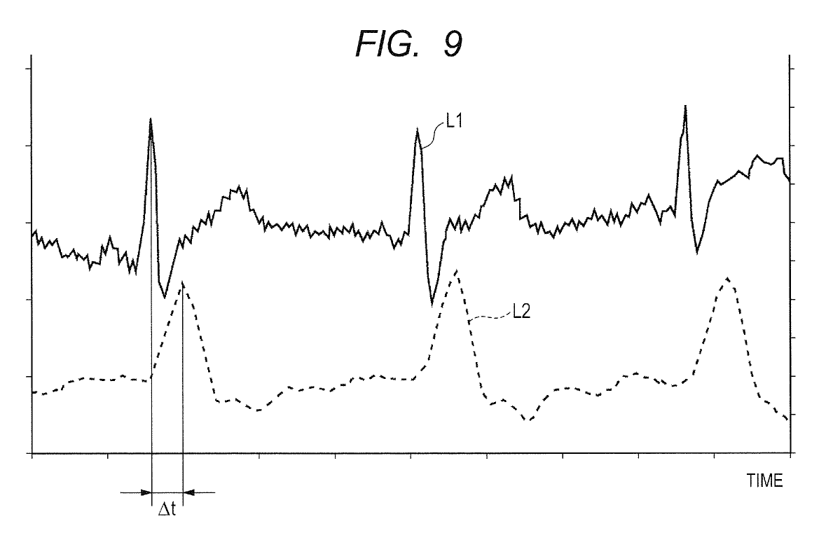

FIG. 9 is a diagram for describing how the condition of the subject' s blood vessels is determined based on heart rate data and pulse data. In FIG. 9, solid line L1 represents heart rate data and broken line L2 represents pulse data. As shown in FIG. 9, pulse data peaks occur after the corresponding peaks of heart rate data. This is due to the time lag between when blood is pumped out from the subject's heart and when the blood reaches terminal portions of the subject's blood vessels. Namely, time difference Δt between a peak of the heart rate data and the corresponding peak of the pulse data approximately equals the time lag between when blood is pumped out from the subject's heart and when the blood reaches terminal portions of the subject's blood vessels.

When the blood vessels of the subject are relatively stiff, the time difference Δt between a peak of the heart rate data and the corresponding peak of the pulse data is smaller than when the blood vessels of the subject are relatively soft. Namely, using heart rate data and pulse data on the subject collected on a regular basis, variation of the condition of the subject's blood vessels can be detected based on time differences between peaks of the heart rate data and the corresponding peaks of the pulse data. The blood vessels of an arteriosclerosis patient are stiffer than the blood vessels of people without arteriosclerosis. Namely, by monitoring variation of the condition of blood vessels of a subject using the heart rate measurement system 210, a sign of the subject's developing arteriosclerosis can be detected.

Next, the flow of processing in the heart rate measurement system 210 will be described below.

FIG. 10 is a flowchart of processing performed in the heart rate measurement system 210. As shown in FIG. 10, first, the heart rate measurement system 210 is turned on (S201). Next, in the detection unit 213, heart rate data is collected based on the potential difference between the intraoral electrode 111 and the hand electrode 112, while also pulse data is collected using the optical sensor 217 (S202). Then, in the detection unit 213, frequency analysis is performed on the heart rate data and pulse data collected (S203) and the heart rate data and pulse data is stored on a time-series basis in a storage medium, for example, a memory included in the detection unit 213 (S204).

Subsequent to S204, whether any part is missing in the heart rate data or pulse data stored is determined in the detection unit 213 (S205). When it is determined in S205 that there is a missing part in the heart rate data or pulse data (YES), the missing part of the heart rate data or pulse data is interpolated by a statistical method for missing-data analysis (S206). The statistical method for missing-data analysis used to analyze the missing part of the heart rate data or pulse data may be a known one. For example, the value of a missing part of the heart rate data or pulse data may be estimated based on the periodicity of the heart rate data or pulse data. The transmission unit 116 transmits the heart rate data and pulse data to the external system 140 (S207). When, in S205, it is determined that no part is missing either in the heart rate data or in the pulse data (NO), processing advances to S207.

Subsequent to S207, in the external system 240, the time difference between a peak of the heart rate data received from the transmission unit 116 and the corresponding peak of the pulse data also received from the transmission unit 116 is calculated and thereby variation of the condition of subject's blood vessels is detected (S208).

When a subject is in a normal state, the time differences between a peak of heart rate data on the subject and the corresponding peak of pulse data on the subject, that is, the time lag between when blood is pumped out from the subject's heart and when the blood reaches terminal portions of the subject's blood vessels varies with the subject's physical constitution (height, weight, arm length, etc.). Therefore, to determine whether the condition of blood vessels of a subject is in a normal range, the subject' s body data is required. By storing the subject's body data in the external system 240 beforehand, the condition of the subject's blood vessels may be determined based on the time difference between a peak of the heart rate data on the subject and the corresponding peak of the pulse data on the subject and also based on the subject's body data. In this way, in S208 shown in FIG. 10, whether the condition of the subject's blood vessels is in a normal range can also be determined besides detecting variation of the condition of the subject's blood vessels.

In S209, besides detecting variation of the condition of the subject's blood vessels using a simple algorithm requiring no body data in the detection unit 213, whether the condition of the subject's blood vessels is in a normal range can also be determined in a simple manner (S209). Either one of S208 performed in the external system 240 and S209 performed in the detection unit 213 may be omitted.

### Third Embodiment

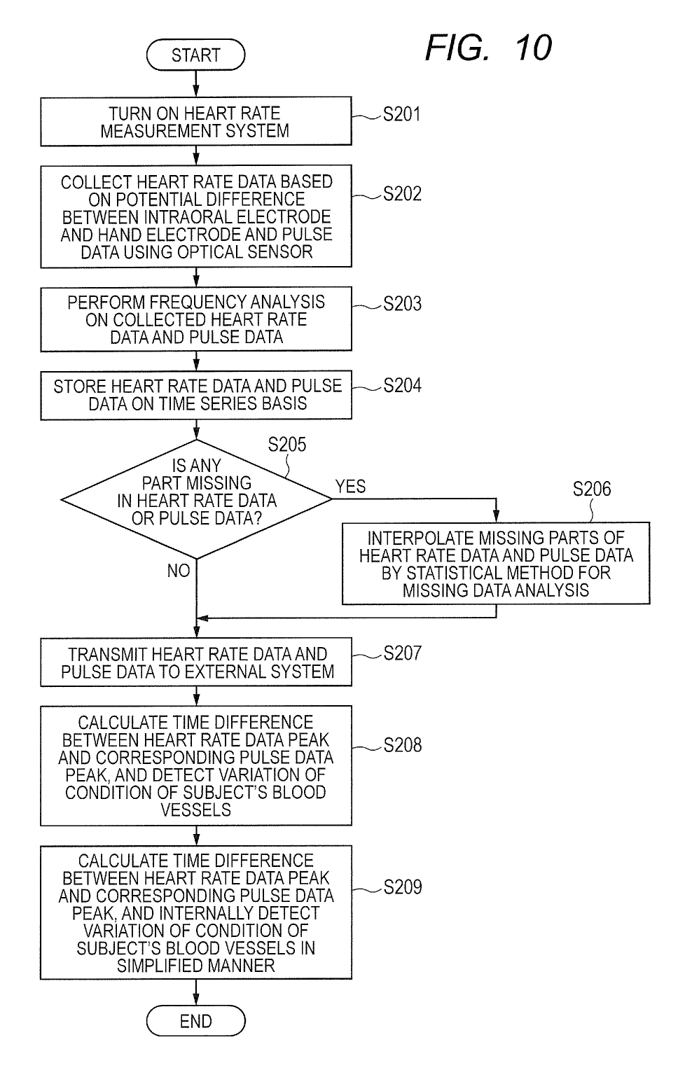

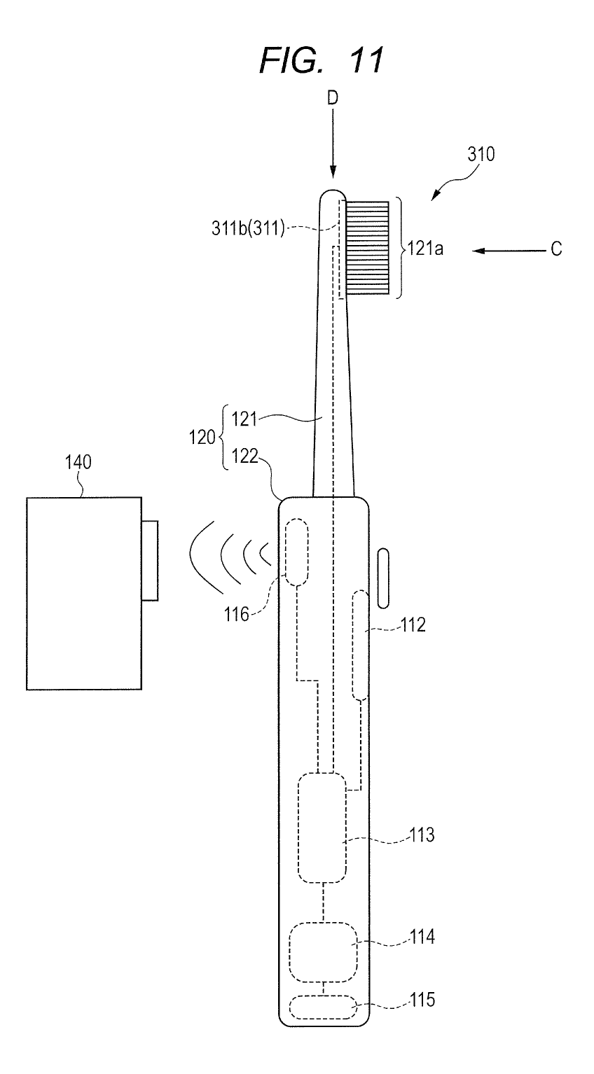

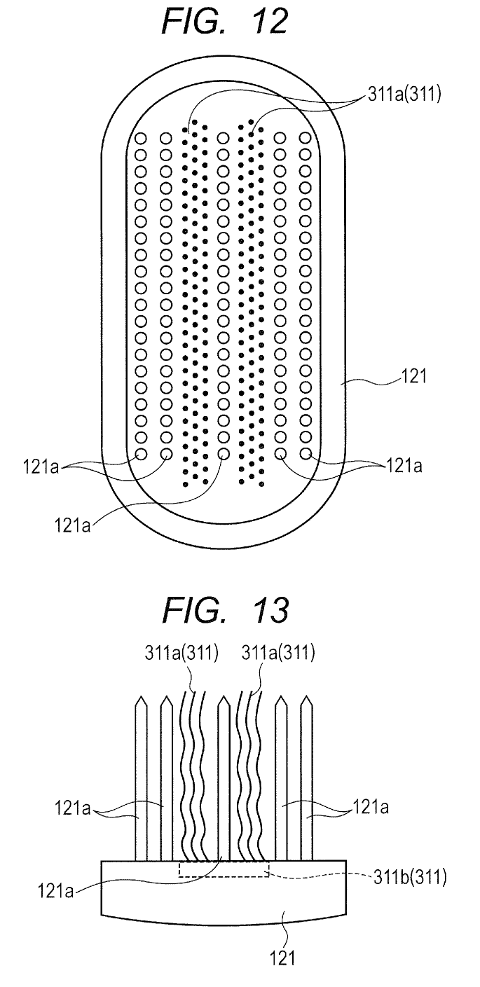

The configuration of a heart rate measurement system 310 according to a third embodiment will be described with reference to FIGS. 11 to 13. In the following, description will be focused on aspects of the third embodiment differing from the first embodiment and the description of configuration aspects similar to those of the first embodiment will be omitted. FIG. 11 is a schematic diagram showing the configuration of the heart rate measurement system 310 according to the third embodiment. FIG. 12 is a view on arrow C in FIG. 11. FIG. 13 is a view on arrow D in FIG. 11.

As shown in FIGS. 11 to 13, unlike the intraoral electrode 111 (see FIGS. 2 and 3) included in the heart rate measurement system 110 according to the first embodiment, the intraoral electrode 311 included in the heart rate measurement system 310 includes conductive fiber bundles 311a implanted along the brush hair 121a. The conductive fiber bundles 311a may be formed of, for example, fibers of polyester or nylon added to by conductive material, for example, conductive ceramics or carbon, or fibers coated with silver. When silver-coated fibers are used, the brush hair 121a will become antibacterial. The conductive fiber bundles 311a are coupled to an electrode plate 311b placed under the brush hair 121a implanted in the head portion 121.

The saliva present in the oral cavity of a human contains an electrolytic material. When the conductive fiber bundles 311a are pressed against an intraoral region of a subject, the saliva present in the oral cavity of the subject is positively led to the conductive fiber bundles 311a by capillary action. Namely, with the conductive fiber bundles 311a coupled to the intraoral electrode 311, the saliva present in the oral cavity of the subject positively promotes the electric contact between an intraoral region of the subject and the intraoral electrode 311, so that the electric contract can be kept in favorable condition. Also, even when the conductive fiber bundles 311a are pressed against teeth of the subject, electric conduction between the conductive fiber bundles 311a and the intraoral skin, for example, a gum portion of the subject is maintained via the saliva, so that heart rate data on the subject can be collected.

Furthermore, when the toothbrush 120 including the heart rate measurement system 310 is an electric toothbrush, the heart rate measurement system 310 can collect heart rate data on a subject while the subject is brushing the teeth. To collect heart rate data on a subject, the heart rate measurement system 310 requires the subject to stay still for a predetermined amount of time (e.g., 5 to 10 seconds) with the intraoral electrode 311 kept in contact with an intraoral region, for example, a gum portion of the subject. Therefore, when the heart rate measurement system 310 is installed in a common non-electric toothbrush, heart rate data collection in a stable state is not possible since the subject keeps moving the arm during tooth brushing. When, on the other hand, the heart rate measurement system 310 is installed in an electric toothbrush, the subject is almost not required to move the arm during tooth brushing. Therefore, the subject can collect the heart rate data during tooth brushing.

When the toothbrush 120 is an electric toothbrush, the grip 122 includes a built-in motor for driving the head portion 121. When the electric toothbrush operates, the motor generates vibration noise. Therefore, to collect heart rate data on a subject during tooth brushing, it may be advisable to make prior arrangement in the detection unit 113 to remove the component of motor vibration noise from the heart rate data collected from the subject. The component of motor vibration noise can be known, for example, from the design specifications of the motor and can then be pre-stored in the detection unit 113. With such arrangement made, heart rate data can be collected from a subject with high accuracy even while the subject is brushing the teeth.

As described above, the saliva present in the oral cavity of a subject is positively led to the conductive fiber bundles 311a by capillary action. Therefore, referring to FIG. 2, the possibility of saliva flowing down from the head portion 121 of the toothbrush 120 to reach the grip 122 increases. The saliva flowing down from the head portion 121 and reaching the grip 122 may cause a short circuit between the intraoral electrode 311 and the hand electrode 112 via the saliva. To prevent such a short circuit, the heart rate measurement system 310 may include a water path for discharging saliva.

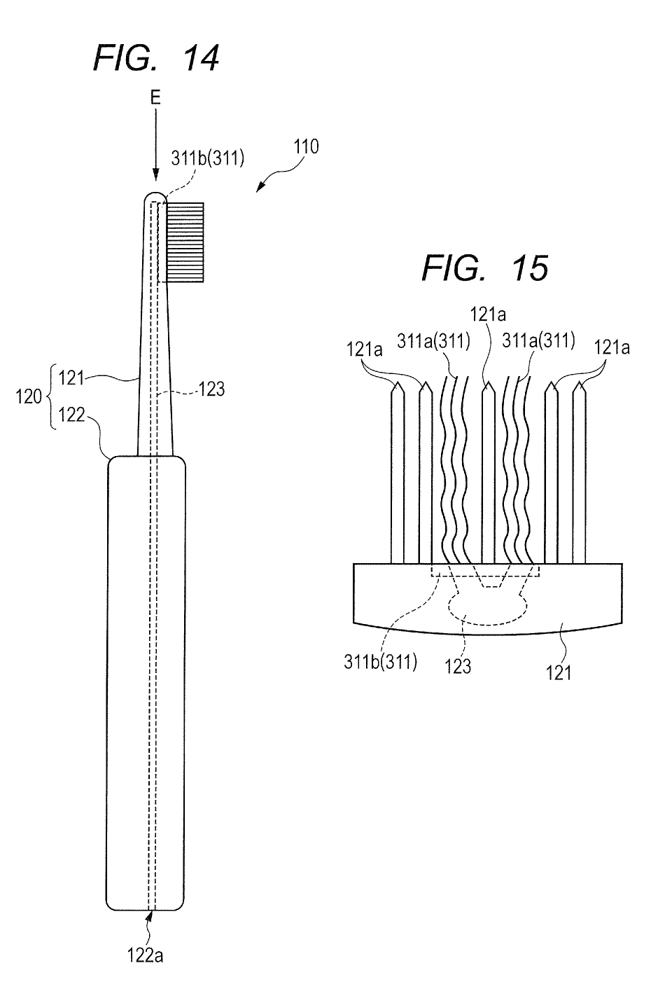

FIG. 14 is a schematic diagram showing a water path 123 included in the heart rate measurement system 310. FIG. 15 is a view on arrow E in FIG. 14. As shown in FIG. 14, the water path 123 communicates from inside the head portion 121 into the grip 122. As shown in FIG. 15, the conductive fiber bundles 311a are coupled to the water path 123. The saliva led from inside the oral cavity of a subject to the conductive fiber bundles 311a by capillary action is discharged from a bottom portion 122a of the grip 122 via the water path 123. Including the water path 123 for discharging saliva in the heart rate measurement system 310 reliably inhibits electric conduction via saliva between the intraoral electrode 311 and the hand electrode 112. Instead of discharging saliva from the bottom portion 122a of the grip 122 (see FIG. 14), a waste tank may be detachably coupled to the water path 123 to allow saliva to pool in the waste tank while the subject is brushing the teeth.

The invention made by the present inventors has been specifically described based on embodiments of the invention, but the invention is not limited to the above-described embodiments and can be modified in various ways without departing from the spirit and scope of the invention.

What is claimed is:

1. A heart rate measurement system comprising:
an intraoral electrode installed in a head portion where brush hair is implanted of a toothbrush, the intraoral electrode being for coming into contact with an intraoral region of a subject;
a hand electrode installed in a grip of the toothbrush so as to come into contact with a hand of the subject; and
a detection unit for detecting a heart rate of the subject,
wherein the detection unit derives, based on signals from the intraoral electrode and the hand electrode, a potential difference between the intraoral region and the hand of the subject and, based on the potential difference derived, collects heart rate data on the subject.

2. The heart rate measurement system according to claim 1, further comprising an optical sensor installed in the grip, the optical sensor being positioned to come into contact with a finger of the subject and including a light source and a light receiving element, the light receiving element detecting an amount of light reflected, after being emitted from the light source to a blood vessel of the finger, from the blood vessel,
wherein the detection unit collects heart rate data on the subject based on the amount of reflected light detected by the optical sensor.

3. The heart rate measurement system according to claim 1,
wherein the hand electrode comprises a plurality of mutually insulated split electrodes, and
wherein the detection unit collects heart rate data on the subject based on, out of signals received from the split electrodes, a signal from which a largest potential difference between the intraoral region and the hand has been derived.

4. The heart rate measurement system according to claim 1,
wherein, when any part is missing in the heart rate data collected, the detection unit interpolates the missing part by a statistical method for missing-data analysis.

5. The heart rate measurement system according to claim 1,
wherein the detection unit detects a variation of condition of a blood vessel of the subject based on a time difference between a peak of heart rate data on the subject and a corresponding peak of pulse data on the subject.

6. The heart rate measurement system according to claim 2, further comprising:
an external system for analyzing heart rate data and pulse data; and
a transmission unit for transmitting the heart rate data and the pulse data collected by the detection unit to the external system,
wherein, in the external system, a variation of condition of a blood vessel of the subject is detected based on a time difference between a peak of heart rate data received from the transmission unit and a corresponding peak of pulse data received from the transmission unit.

7. The heart rate measurement system according to claim 5,
wherein, by having body data on the subject stored beforehand, the external system determines condition of a blood vessel of the subject based on the time difference and the body data.

8. The heart rate measurement system according to claim 1,
wherein the intraoral electrode is installed on a side of the head portion opposite to the other side where the brush hair is implanted.

9. The heart rate measurement system according to claim 1,
wherein the intraoral electrode includes a conductive fiber bundle implanted, along with the brush hair, in the head portion.

10. The heart rate measurement system according to claim 9,
wherein the conductive fiber bundle is coupled to a water path communicating from inside the head portion into the grip.

11. The heart rate measurement system according to claim 1,
wherein the head portion and the grip are coated with a water-repellent material.

12. The heart rate measurement system according to claim 8,
wherein the toothbrush is an electric toothbrush,
wherein the electric toothbrush includes a motor built into the grip and, by driving the head portion using the motor, collects heart rate data on the subject while the subject is brushing the subject's teeth, and
wherein the detection unit performs processing to remove a pre-stored vibration noise component of the motor from the collected heart rate data.

13. A heart rate measurement method,
wherein a toothbrush including a head portion with brush hair implanted therein and a grip is used, the head portion including an intraoral electrode for coming into contact with an intraoral region of a subject, the grip including a hand electrode for coming into contact with a hand of the subject, and
wherein a potential difference between the intraoral region and the hand of the subject is derived based on signals from the intraoral electrode and from the hand electrode and heart rate data on the subject is collected based on the derived potential difference.

14. The heart rate measurement method according to claim 13,
wherein, when any part is missing in the heart rate data, the missing part is interpolated by a statistical method for missing-data analysis.

15. The heart rate measurement method according to claim 13,
wherein the toothbrush includes an optical sensor installed in the grip and positioned to come into contact with a finger of the subject, the optical sensor including a light source and a light receiving element and measuring, using the light receiving element, an amount of light reflected after being emitted from the light source to a blood vessel of the finger,
wherein pulse data on the subject is collected based on the amount of reflected light detected by the optical sensor, and
wherein a variation of condition of a blood vessel of the subject is detected based on a time difference between a peak of the heart rate data and a corresponding peak of the pulse data.

16. The heart rate measurement method according to claim 15,
wherein the condition of a blood vessel of the subject is determined based on the time difference and body data on the subject.
